(19) United States
(12) Patent Application Publication
(10) Pub. No.: US 2023/0301977 A1
(43) Pub. Date: Sep. 28, 2023

(54) WOUND HEALING MODEL

(71) Applicants: Agency for Science, Technology and Research, Singapore (SG); Nanyang Technological University, Singapore (SG)

(72) Inventors: David Becker, Singapore (SG); Jiah Shin Chin, Singapore (SG); Leigh Madden, Singapore (SG); Li Ling Mandy Tan, Singapore (SG)

(21) Appl. No.: 18/245,423

(22) PCT Filed: Sep. 16, 2021
(86) PCT No.: PCT/SG2021/050564

§ 371 Date: Mar. 15, 2023

(30) Foreign Application Priority Data
Sep. 16, 2020 (SG) 10202009068U

(51) Int. Cl. A61K 31/4545; A61L 27/24; A61P 17/02

(52) U.S. Cl. CPC A61K 31/4545; A61L 27/24; A61P 17/02; A61L 2300/412

(57) ABSTRACT

The invention relates generally to the field of wound healing. In one embodiment is a method for generating a delayed wound model in an animal, the method comprising contacting a wound with a composition comprising an electrospun scaffold, wherein the scaffold is made from 80% PCL and 20% rat tail collagen and has been soaked in a biofilm conditioned media from Staphylococcus aureus or a small molecular drug FK866.

### FIELD OF INVENTION

The invention relates generally to the field of wound healing. In particular, the specification teaches a method for generating a delayed wound healing model in an animal and compositions thereof.

### BACKGROUND

Wound healing refers to the process by which damaged tissues are repaired or replaced. It usually involves four stages: haemostasis, inflammation, proliferation and remodelling. Acute wound healing typically proceeds rapidly in healthy individuals and there is little need to promote it. In elderly or diabetic patients, healing is often slow and can become chronic. This represents a major unmet clinical need.

Most animal wounds heal rapidly without any intervention unlike human chronic wounds which can remain open for months or years. Whilst diabetic animals show delayed wound healing, this is only slowed by 1-2 day in rats and 3-4 days in pigs. As a result, there are no good animal models to test novel therapeutics for the treatment of chronic wounds. The lack of a good model is thought to underlie the failure of many wound healing therapeutics when they are tested in humans.

Accordingly, it is generally desirable to overcome or ameliorate one or more of the above mentioned difficulties.

### SUMMARY

Disclosed herein is a method for generating a wound model in an animal, the method comprising contacting a wound with a composition comprising a scaffold and a senescence-inducing agent.

Disclosed herein is a wound animal model generated according to a method as defined herein.

Disclosed herein is a method of identifying a candidate therapeutic agent for treating a wound, the method comprising a) generating a wound model in an animal according to a method as defined herein, and b) contacting the wound model with the candidate therapeutic agent.

Disclosed herein is a composition for inducing the formation of a wound model in an animal, the composition comprising a scaffold and a senescence-inducing agent.

Disclosed herein is a dermal substitute for inducing the formation of a wound model in an animal, the dermal substitute comprising a scaffold and a senescence-inducing agent.

### BRIEF DESCRIPTION OF DRAWINGS

Embodiments of the present invention are hereafter described, by way of non-limiting example only, with reference to the accompanying drawings in which:

FIG. 1: Schematic of technology—creation of the perturbed wound healing model.

FIG. 2: Timeline comparison between wound models in rats. Drastic reduction in duration of model generation when comparing diabetic wound model against perturbed wound model, this translates to lower animal usage, reduced animal distress and less man/research hours.

FIG. 3: Generation of perturbed healing model in rats.

FIG. 4: Macroscopic analysis of wounds in rats comparing across three models—acute, diabetic and perturbed wound healing.

FIG. 5: Histological analysis of wounds in rats comparing across three models—acute, diabetic and perturbed wound healing.

FIG. 6: Generation of perturbed healing model in pigs

FIG. 7: Macroscopic analysis of wounds generated with acute and perturbed healing model in pigs.

FIG. 8: Histological analysis of wounds generated with acute and perturbed healing model in pigs.

FIG. 9: Histological images of senescent cells (beta-galactosidase) present in perturbed wounds at day 20 post scaffold removal. Senescent cells can be seen at the margin of the wounds, throughout the granulation tissue and along the walls of blood vessels.

FIG. 10. Images showing progression of wounds with scaffold placement for 5 or 10 days in situ. B) Schematic illustrating measurement of nascent epidermal tongue. C) Panel depicting representative H&E images of acute and perturbed wounds. Re-epithelialization from the wound edge is traced by black lines. Scale bar=2000 μm. D) Quantitative evaluation of extent of re-epithelialization (n=1, n represents 1 biological replicate). Re-epithelialization was analyzed by expressing the total re-epithelialization distance as a percentage of the width of the wound bed.

FIG. 11. Analysis of epidermal tongue thickness. A) Schematic illustrating area and length measurement of nascent epidermal tongue. B) Panel depicting representative H&E images of acute and perturbed wounds. Area of epidermal tongue is outlined by black dots. Migration of epidermal tongue from the wound edge is traced by black lines. Scale bar=1000 μm. C) Quantitative evaluation of the average thickness of epidermal tongue (n=1, n represents 1 biological replicate). Thickness of epidermal tongue was analyzed by taking the area over the re-epithelialized distance.

FIG. 12. Analysis of inflammatory condition in acute and perturbed wounds. A) Schematic illustrating regions of interest selected for quantitative measurement. B) Panel depicting representative H&E images of acute and perturbed wounds (Selected regions outlined in black lines in A). Scale bar=50 μm. C) Quantitative evaluation of histological inflammation scores of acute and perturbed wound beds (n=1, n represents 1 biological replicate).

FIG. 13. Macroscopic and histological assessment of acute and perturbed wounds. A) Macroscopic images showing progression of wound closure for acute and perturbed wounds. B) Schematic illustrating measurement of nascent epidermal tongue. C) Panel depicting representative H&E images of acute and perturbed wounds. Re-epithelialization from the wound edge is marked by black lines. Scale bar=2000 μm. D) Quantitative evaluation of extent of re-epithelialization (n=3, n represents 1 biological replicate). Re-epithelialization was analyzed by expressing the total re-epithelialization distance as a percentage of the width of the wound bed.

FIG. 14. Analysis of epidermal tongue thickness and Cx43 expression. A) Schematic illustrating area and length measurement of nascent epidermal tongue. B) Panel depicting representative H&E and immunofluorescent staining (Cx43 shown by punctate staining) images from acute and perturbed wounds. Area of epidermal tongue is outlined by black dots. Migration of epidermal tongue from the wound edge is marked by black lines. Black dotted square depicts region where corresponding confocal images were taken. Epithelial tongue is marked by white dotted lines. Black scale bar=1000 μm, white scale bar=40 μm. C) Quantitative evaluation of the average thickness of epidermal tongue (n=3, n represents 1 biological replicate). Thickness of epidermal tongue was analyzed by taking the area over the re-epithelialized distance. D) Quantification of Cx43 expression at leading epidermal tongue (n=3, n represents 1 biological replicate).

FIG. 15. Analysis of inflammatory condition in acute and perturbed wounds. A) Schematic illustrating regions of interest selected for quantitative measurement. B) Panel depicting representative H&E images of acute and perturbed wound beds (Selected regions outlined in black lines in A). Scale bar=50 μm. C) Quantitative evaluation of histological inflammation scores of acute and perturbed wound beds (n=3, n represents 1 biological replicate). D) Panel depicting representative H&E images of acute and perturbed wound edges (Selected regions outlined in black dots in A). Scale bar=50 μm. E) Quantitative evaluation of histological inflammation scores of acute and perturbed wound edges (n=3, n represents 1 biological replicate).

FIG. 16. Analysis of collagen content in acute and perturbed wounds. A) Schematic illustrating regions of interest selected for quantitative measurement. B) Panel depicting representative Masson's Trichrome images of acute and perturbed wound beds (Selected regions outlined in black lines in A). Scale bar=50 μm. C) Quantitative evaluation of collagen deposition in acute and perturbed wound beds (n=3, n represents 1 biological replicate). D) Panel depicting representative H&E images of acute and perturbed wound edges (Selected regions outlined in black dots in A). Scale bar=50 μm. E) Quantitative evaluation of collagen loss at acute and perturbed wound edges (n=3, n represents 1 biological replicate).

FIG. 17. Analysis of senescent cell population in acute and perturbed wounds. A) Schematic illustrating regions of interest selected for quantitative measurement. B) Panel depicting representative X-gal images of acute and perturbed wound beds (Selected regions outlined in black lines in A). Scale bar=50 μm. C) Quantitative evaluation of X-gal positive cells in acute and perturbed wound beds (n=3, n represents 1 biological replicate). D) Panel depicting representative H&E images of acute and perturbed wound edges (Selected regions outlined in black dots in A). Scale bar=50 μm. E) Quantitative evaluation of Xgal positive cells at acute and perturbed wound edges (n=3, n represents 1 biological replicate).

FIG. 18. Summary of features observed in perturbed wound model.

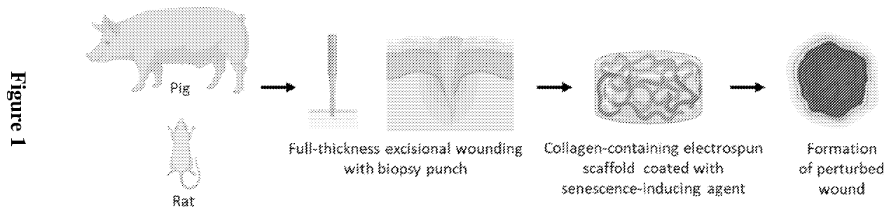

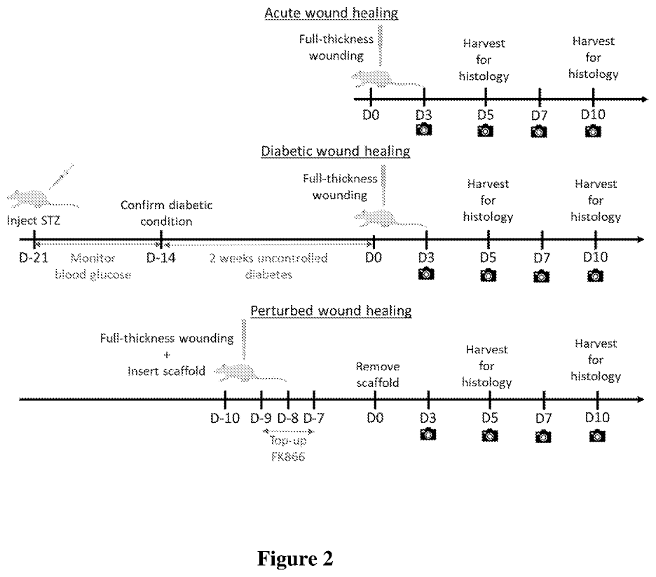

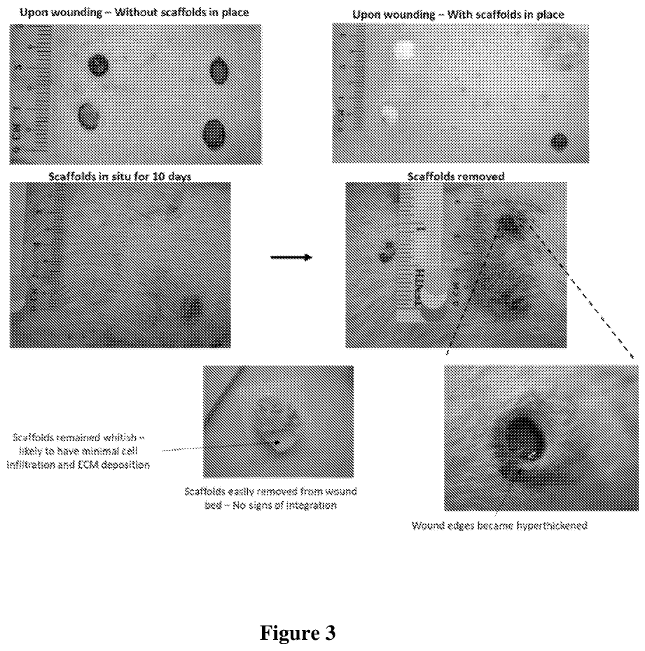

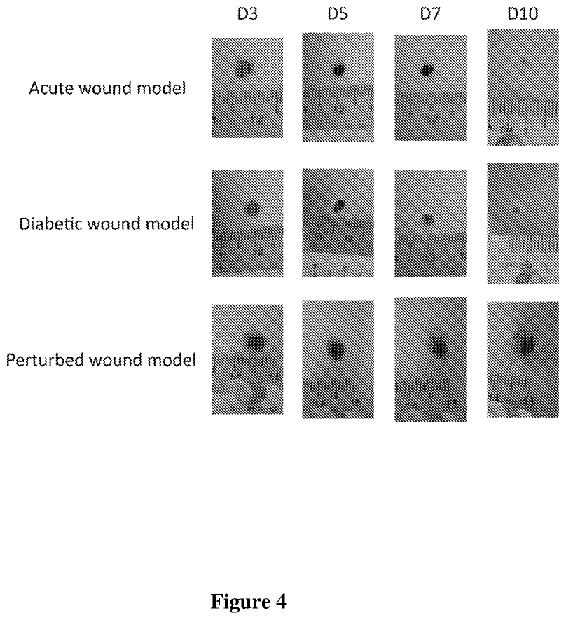

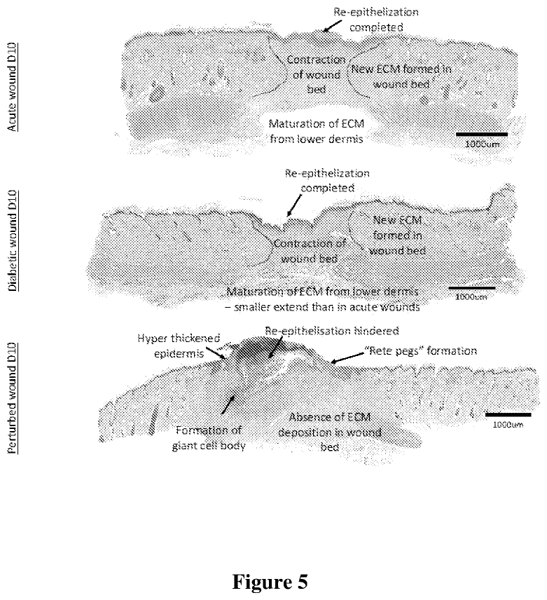

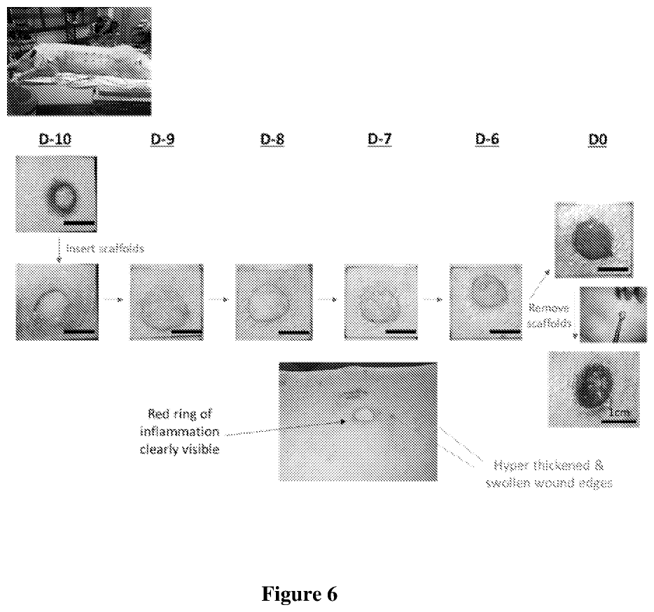

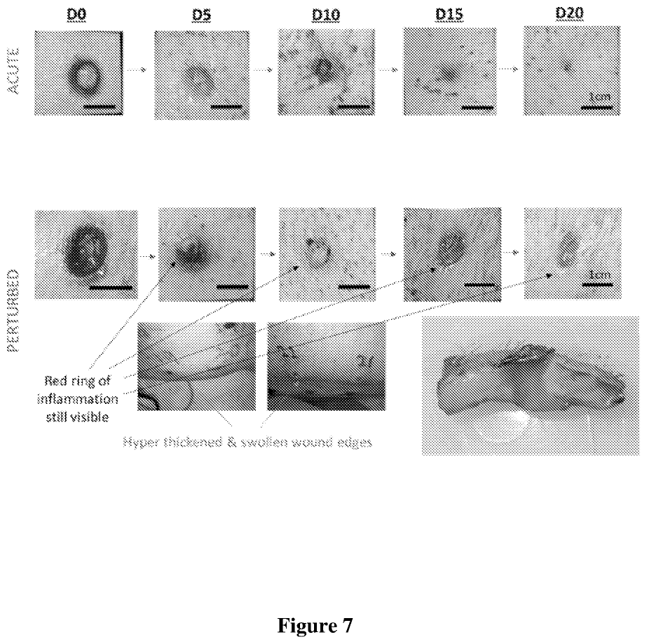

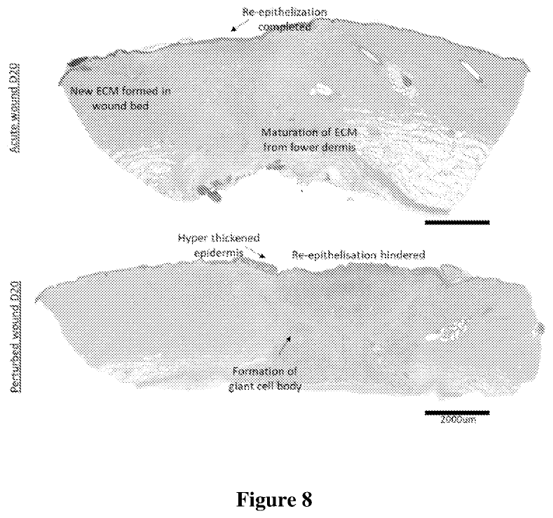

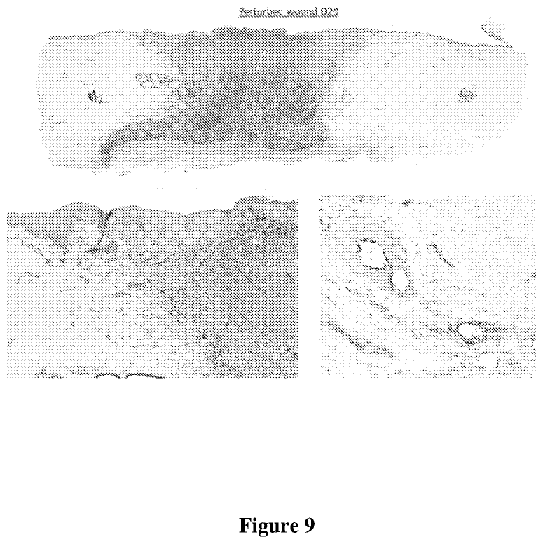

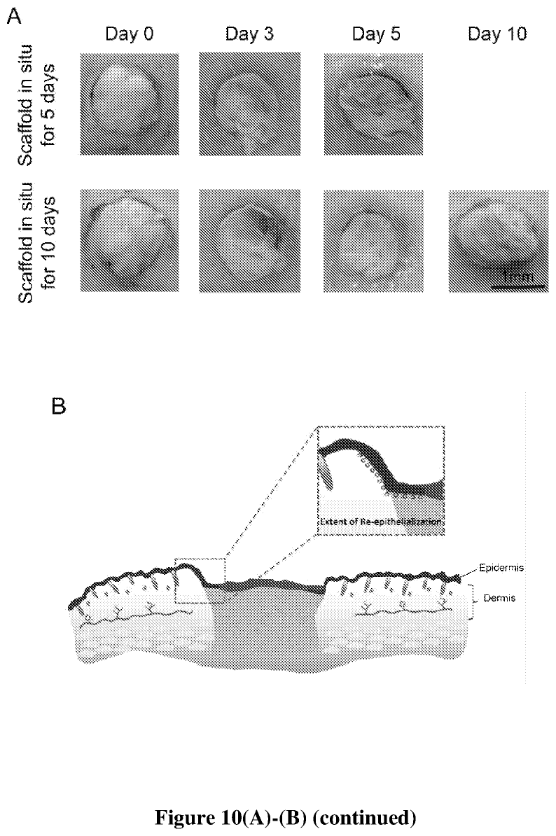

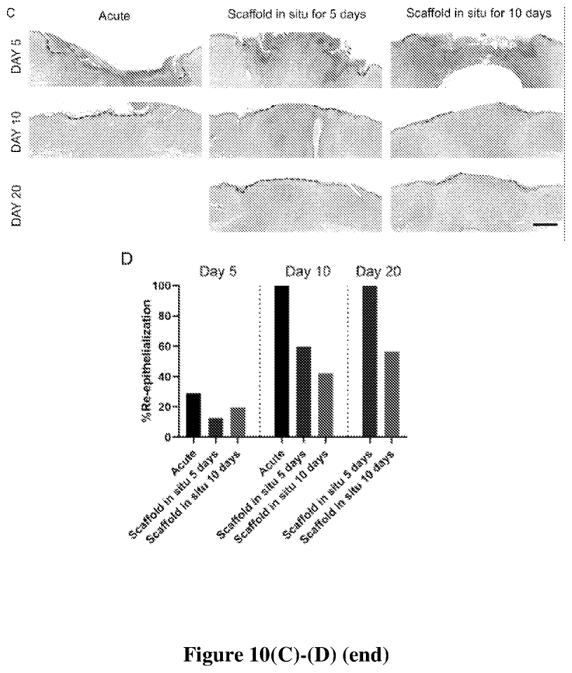

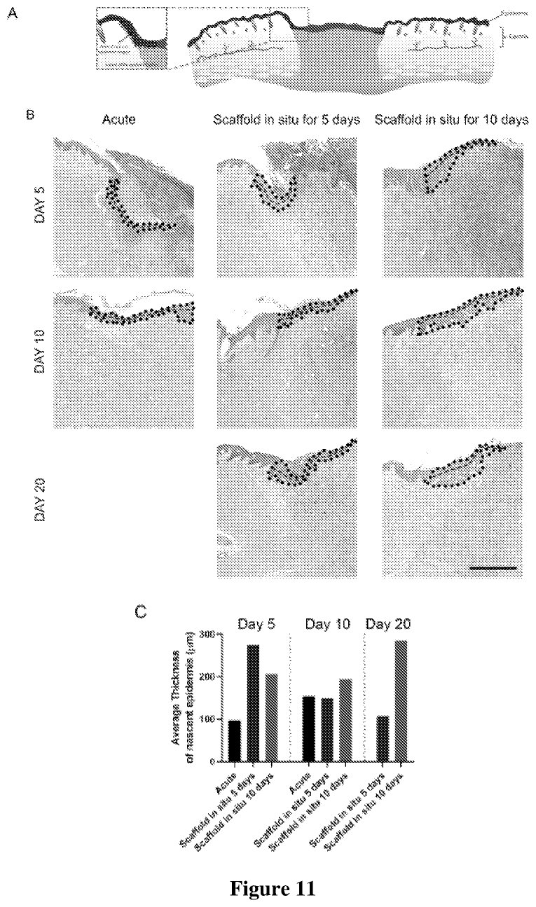

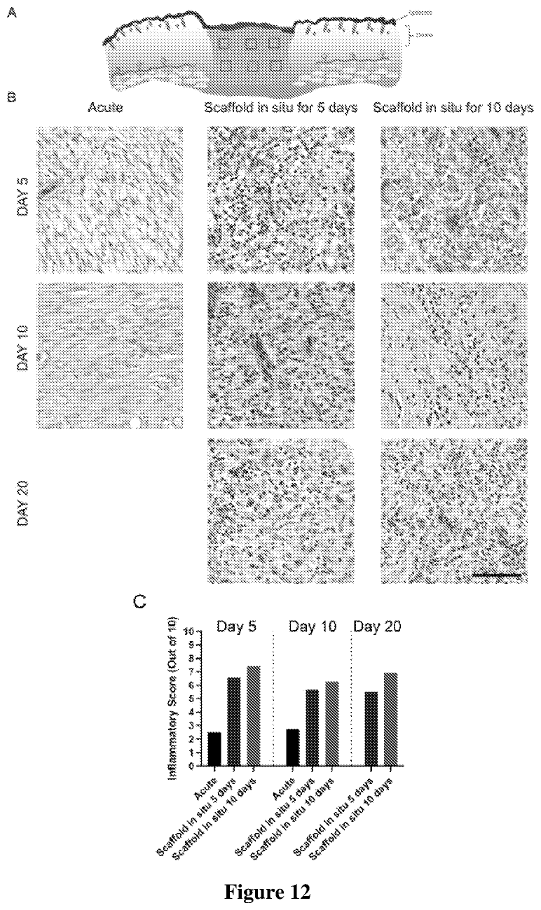

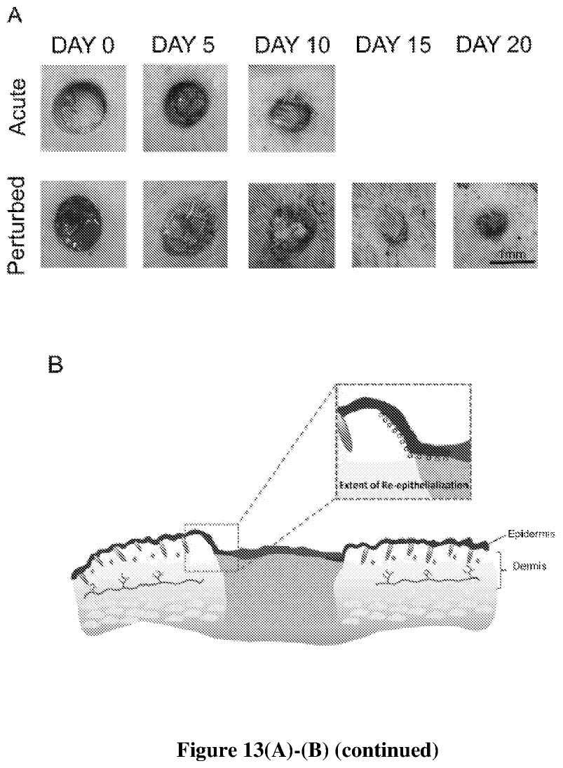

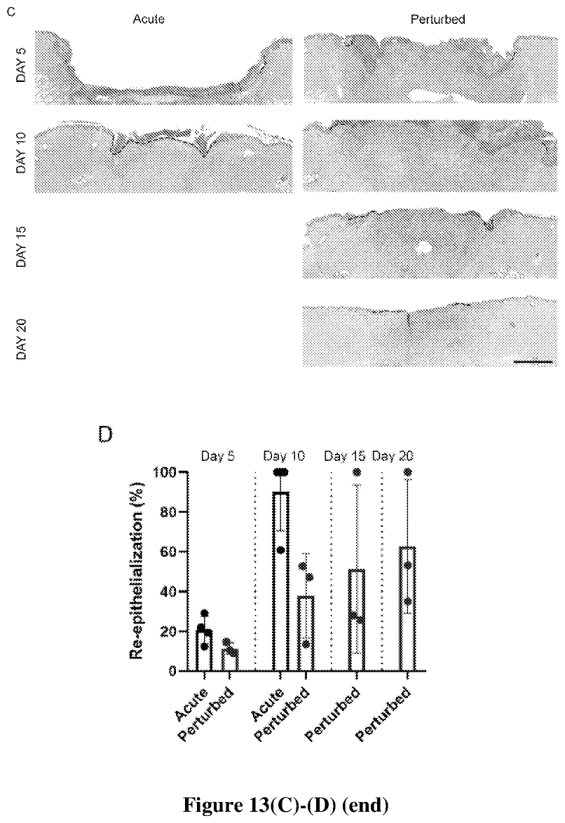

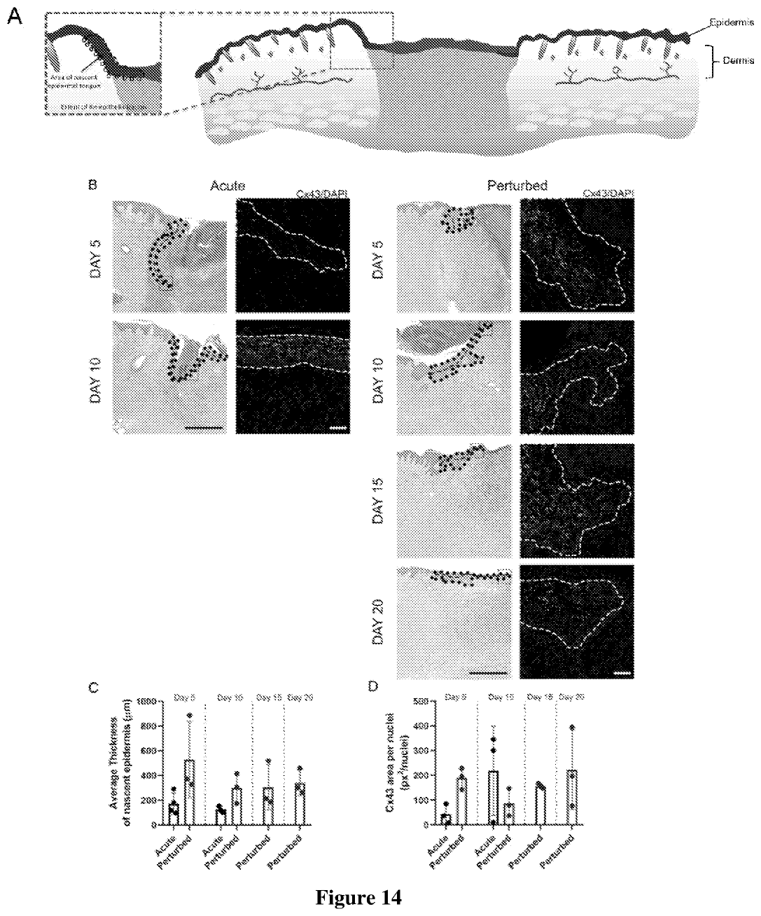

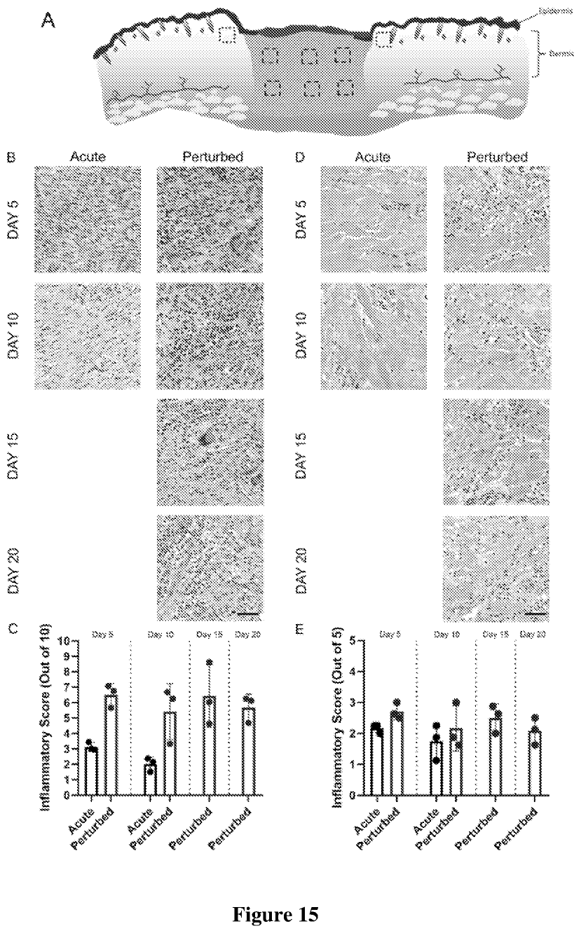

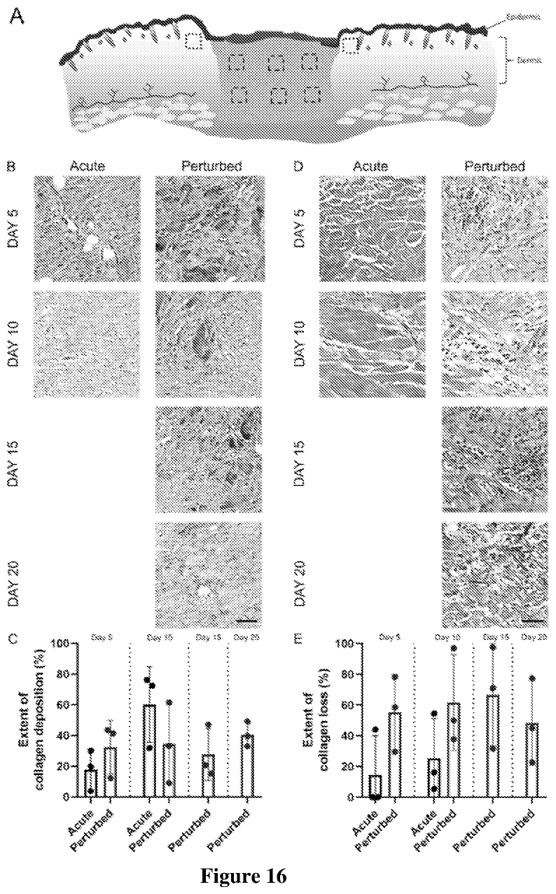

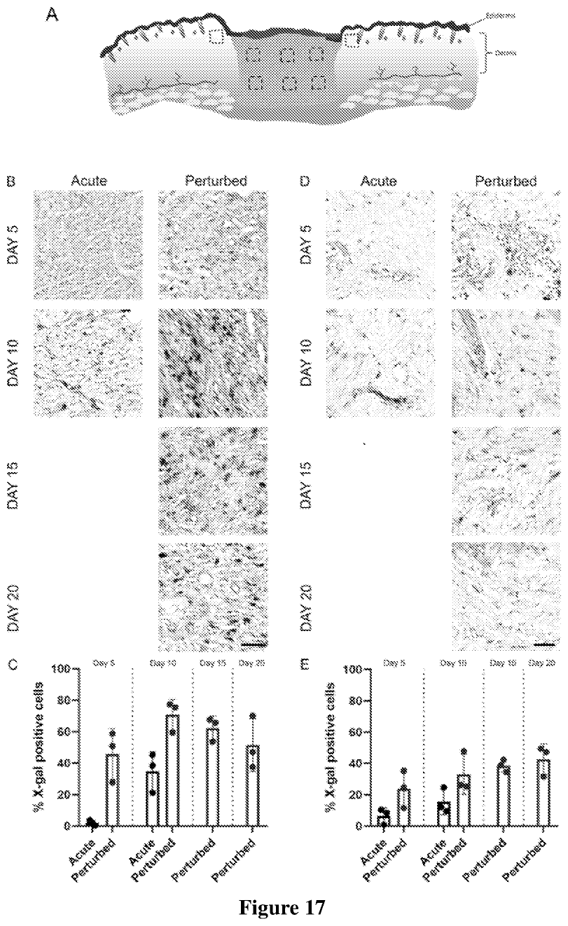

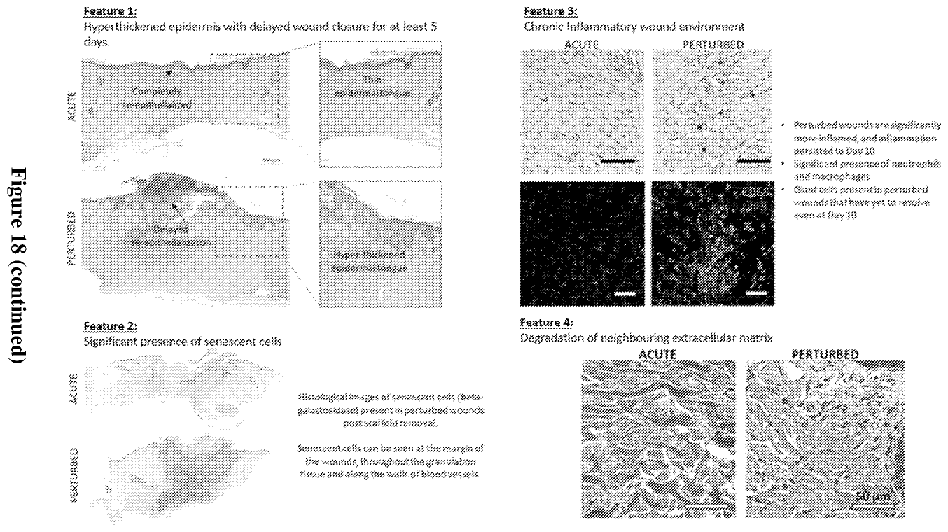

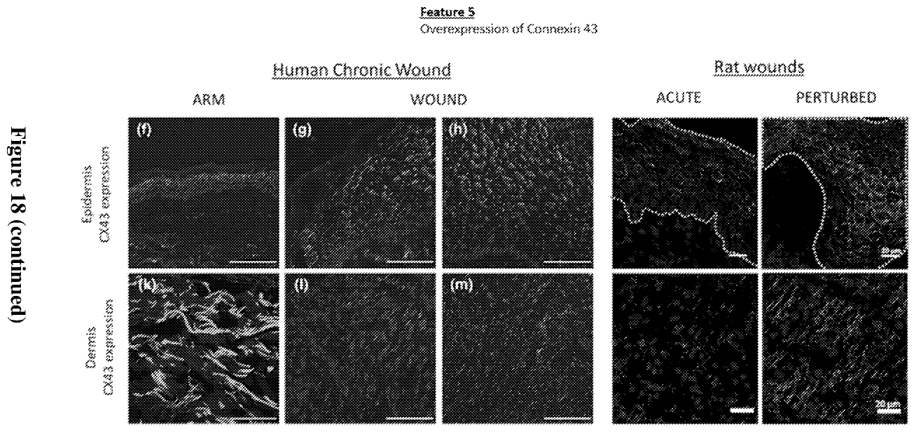

### DETAILED DESCRIPTION

The present disclosure teaches a method of generating a wound model in an animal.

Disclosed herein is a method for generating a wound model in an animal, the method comprising a) contacting a wound with a composition comprising a scaffold and a senescence-inducing agent.

The method may comprise contacting for a sufficient time and under conditions to generate the wound model in the animal.

In one embodiment, there is provided a method for generating a wound model in an animal, the method comprising a) contacting a wound with a composition comprising one or more polymers and a senescence-inducing agent.

Without being bound by theory, the inventors have developed a perturbed wound healing model in both rats and pigs. This model is able to recapitulate several features of human chronic wounds such as chronic inflammation, hyper thickened non-migratory epidermis, senescent fibroblasts and endothelial cells. Unlike diabetic rats and pigs, where healing is delayed for a maximum of 2-4 days, these wounds are still not closed after 10 and 30-35 days respectively. More importantly, this model when compared to the current FDA-approved Streptozotocin-induced diabetic model of wound healing is a refinement of the standard practice. In particular, the animals undergo minimal pain and distress compared to the induction of diabetes and associated side effects while achieving the characteristics of a perturbed wound.

As used herein, a “wound” is a region of damaged tissue. The damaged tissue may be due to trauma (e.g. mechanical, such as wounds from surgical procedures (including incisions, tooth extractions, or other surgical procedures), wounds from accidents), infection, and/or inflammation. Examples of wounds include: wounds resulting from an incision such as cutting instrument (e.g. incision in surgery), lacerations (typically caused by blunt or broken instrument), puncture wounds, abrasions, burn wounds resulting from exposure to heat, electricity, radiation (for example, sunburn and laser surgery); wounds resulting from surgical procedures such as tooth extraction; caustic chemicals; skin wounds due to aging or the environment, including for example split, dry skin; ulcers (lesion on the surface of the skin or a mucous surface); wounds in subjects suffering from Diabetes Mellitus or other conditions such as ischemia, including foot injuries due to numbness caused by nerve damage (diabetic neuropathy) and low blood flow to the legs and feet, foot ulcers, decubitus wounds, decubitus (bedsores).

In one embodiment, the method comprises generating a wound in the animal prior to step (a). The wound may be a fresh wound. The wound may be a skin wound. The wound may be a full-thickness skin wound. In one embodiment, the wound is a skin-puncture wound or excision wound. The wound may be generated by a punch biopsy. The punch biopsy may be a full-thickness punch biopsy. The punch biopsy may, for example, be a circular punch biopsy that is about 6 mm in diameter in rats or about 10 mm to 12 mm in diameter in pigs.

In one embodiment, the wound model is a perturbed wound model. The perturbed wound model may share many of the features of human chronic wounds

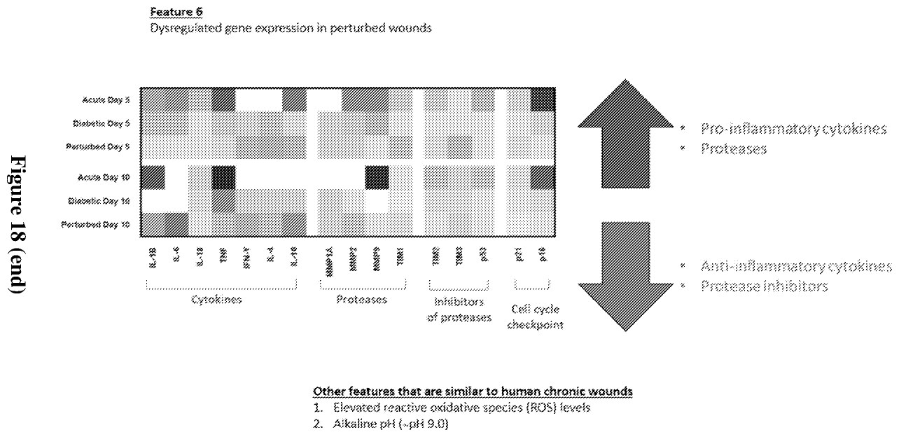

The term “Perturbed wound” as used herein may refer to a wound that fail to proceed through the normal phases of wound healing in an orderly and timely manner. A “perturbed wound model” or “perturbed wound healing model” tries to recapitulate several features of chronic wounds in an animal model. These features may include, for example, chronic inflammation, hyper thickened non-migratory epidermis, senescent fibroblasts and endothelial cells, over expression of the gap junction protein Cx43, elevated ROS levels and high pH (see, for example, FIG. 18).

In one embodiment, the wound model is a non-human model. The non-human model may be used to understand chronic wound healing in human.

In one embodiment, the wound model is a perturbed wound model. In one embodiment, the wound model is a perturbed wound healing model.

The term “healing” in respect to a wound or a skin damage refers to a process to repair a wound, or to repair the skin damage.

As used herein, the term “contacting” may refer to bringing about direct contact between a composition and a wound in an animal such that they are in immediate proximity or association with each other. Contacting can occur, for example, as a result of applying or implanting the composition.

In one embodiment, the composition is capable of inducing a foreign body reaction (FBR) within the wound of an animal.

The term “scaffold” refers to a three-dimensional structure that can be formed by a network of one or more polymers which can be inserted into a wound on an animal. The scaffold may provide sufficient mechanical strength or stiffness to resist wound contraction without being forced out of the wound. The scaffold may also allow structural integrity to be maintained so that it can be removed cleanly from the wound.

In one embodiment, the composition comprises a scaffold formed by the one or more (such as one, two three, four or more) polymers. The scaffold may, for example, be inserted into a wound (such as one generated by a punch biopsy). The scaffold may be an electrospun scaffold. The scaffold may, alternatively, be generated by other fabrication methods such as 3D printing, solvent casting and freeze drying. In one embodiment, the composition comprises a solid scaffold, such as a solid electrospun scaffold. The composition (or scaffold) may be a circular disc or rod that can be inserted into a wound.

The term “electrospinning” generally refers to techniques that make use of a high-voltage power supply, a spinneret (e.g., a hypodermic needle), syringe pump, and an electrically conductive collector plate or liquid bath (e.g., aluminium foil or 100% ethanol) that can be used to prepare an electrospun scaffold. To perform the electrospinning process, an electrospinning liquid (i.e. a melt or solution of the desired materials, dissolved in solvents, that will be used to form the fibres) is generally first loaded into a syringe and is then fed at a specific rate set by a syringe pump.

As the liquid is fed by the syringe pump with a sufficiently high voltage, the repulsion between the charges immobilized on the surface of the resulting liquid droplet overcomes the confinement of surface tension and induces the ejection of a liquid jet from the orifice. The charged jet then goes through a whipping and stretching process, and subsequently results in the formation of uniform fibers. Further, as the jet is stretched and the solvent is evaporated, the diameters of the fibres can then be continuously reduced to a desired scale, for example micrometers or nanometers and, under the influence of an electrical field, the fibres can subsequently be forced to travel towards a collector.

In one embodiment, the one or more polymers is selected from the group consisting of polycaprolactone (PCL), collagen, fibroin, gelatin, poly(3-hydroxybutyrate-co-3-hydroxyvalerate) (PHBV), poly(lactic-co-glycolic acid) (PLGA), polylactic acid (PLA) and poly(glycolic acid)(PGA). In one embodiment, the one or more polymers comprises or consists of collagen. In one embodiment, the one or more polymers comprises or consists of PCL.

In one embodiment, the one or more polymers comprises or consists of collagen and a synthetic polymer. The synthetic polymer may be selected from the group consisting of PCL, PLGA, PLA and PGA and any other synthetic polymer that is known in the art.

In one embodiment, the one or more polymers comprises or consists of PCL and collagen. In one embodiment, the one or more polymers comprises or consists of PLGA and collagen. In one embodiment, the one or more polymers comprises or consists PLA and collagen. In one embodiment, the one or more polymers comprises or consists of PGA and collagen.

The composition may comprise about 70%-90% w/w of synthetic polymer. For example, the composition may comprise about 70%, 71%, 72%, 73%, 74%, 75%, 76%, 77%, 78%, 79%, 80%, 81%, 82%, 83%, 84%, 85%, 86%, 87%, 88%, 89%, or 90% w/w synthetic polymer. The composition may comprise about 10-30% w/w collagen. For example, the composition may comprise about 10%, 11%, 12%, 13%, 14%, 15%, 16%, 17%, 18%, 19%, 20%, 21%, 22%, 23%, 24%, 25%, 26%, 27%, 28%, 29% or 30%. In one embodiment, the composition comprises about 80% w/w synthetic polymer and about 20% w/w collagen

The composition may comprise about 70%-90% w/w PCL. For example, the composition may comprise about 70%, 71%, 72%, 73%, 74%, 75%, 76%, 77%, 78%, 79%, 80%, 81%, 82%, 83%, 84%, 85%, 86%, 87%, 88%, 89%, or 90% w/w PCL. The composition may comprise about 10-30% w/w collagen. For example, the composition may comprise about 10%, 11%, 12%, 13%, 14%, 15%, 16%, 17%, 18%, 19%, 20%, 21%, 22%, 23%, 24%, 25%, 26%, 27%, 28%, 29% or 30%. In one embodiment, the composition comprises about 80% w/w PCL and about 20% w/w collagen

In one embodiment, the collagen is a type I collagen. In one embodiment, the collagen is rat collagen. The rat collagen may be rat tail collagen. In one embodiment, the collagen is a rat tail type I collagen.

The term “senescence-inducing agent” may refer to any agent that is capable of inducing senescence in a cell of an animal, which is a process by which a cell ages and permanently stops dividing but does not die. The senescence-inducing agent may include DNA damaging agents, reactive oxygen species generating agents, differentiation agents or ionizing radiation. The senescence-inducing agent may be a biofilm conditioned media (BCM) or a nicotinamide phosphoribosyltransferase inhibitor. The senescence-inducing agent may also be, for example, aphidicolin, bleomycin, cisplatin, doxorubicin, etoposide, mitoxantrone, retinols, hydroxyurea, carboplatin or docetaxel. The senescence-inducing agent may also be any senescence-inducing agent disclosed in Ewald J A et al 2010 or Mikula-Pietrasik, J. et al. 2020.

In one embodiment, the senescence-inducing agent is biofilm conditioned media (BCM). The biofilm conditioned media may be a biofilm conditioned media from bacteria. The bacteria may be any bacteria that can be found in a wound. The biofilm conditioned media may be a biofilm conditioned media from Staphylococcus aureas, Streptococcus pyogenes, Pseudomonas aeruginosa or Enterococcus faecalis.

In one embodiment, the senescence-inducing agent is a nicotinamide phosphoribosyltransferase inhibitor. The term “inhibitor” may refer to any molecule (such as a small molecule or peptide) that is capable of decreasing the activity of nicotinamide phosphoribosyltransferase or decrease the protein level of nicotinamide phosphoribosyltransferase. An inhibitor can also be a molecule (such as a nucleic acid or ribozyme) which decreases the expression of the gene encoding nicotinamide phosphoribosyltransferase.

The nicotinamide phosphoribosyltransferase inhibitor may, for example, be Daporinad (FK866) or CHS-828 (or GMX-1778). The nicotinamide phosphoribosyltransferase inhibitor may be present at a concentration of about 10 μM, 20 μM, 30 μM, 40 μM, 50 μM, 60 μM, 70 μM, 80 μM, 90 μM, 100 μM, 110 μM, 120 μM, 130 μM, 140 μM, 150 μM, 160 μM, 170 μM, 180 μM, 190 μM, 200 μM, 300 μM, 400 μM, 500 μM, 600 μM, 700 μM, 800 μM, 900 μM or 1000 μM.

The nicotinamide phosphoribosyltransferase inhibitor may also be a nucleic acid, such as an antisense RNA or shRNA. The inhibitor may also be a gene-editing composition for knocking out or knocking down nicotinamide phosphoribosyltransferase. Such gene-editing compositions are well known in the art and includes, for example, the CRISPR-Cas gene-editing system, Talen gene-editing systems and Zinc Finger gene-editing system.

In one embodiment, the method comprises providing an animal. The animal may be a mammal. The animal may, for example, be a mouse, rat or a pig.

In one embodiment, the method comprising sealing the wound following step a). The method may comprise sealing the wound with a non-breathable material. The wound may be covered with parafilm, Tegaderm, Opsite film and/or sticky crepe bandage. The sealing of the wound may help to prevent contamination.

In one embodiment, the method comprises removing and/or contacting the wound with the composition once a day over two, three, four, five or more days.

In one embodiment, the method comprises further contacting the wound with the senescence-inducing agent every one, two, three, four, five or more days.

In one embodiment, the method comprises removing the composition from the wound to allow generation of the wound model in the animal.

Disclosed herein is a method for generating a foreign body reaction (FBR) in an animal, the method comprising a) contacting a wound with a composition as defined herein.

Disclosed herein is a wound animal model generated according to a method as defined herein.

In one embodiment, there is provided a wound animal model. The wound animal model may comprise a wound that has been contacted with a composition comprising a scaffold and a senescence-inducing agent. The wound animal model may be suitable for evaluating wound healing, such as perturbed/delayed wound healing.

Disclosed herein is a method of identifying a candidate therapeutic agent for treating a wound, the method comprising a) generating a wound model in an animal according to a method as defined herein, and b) contacting the wound model with the candidate therapeutic agent. The method may comprise testing the candidate therapeutic agent on the wound to determine whether the candidate therapeutic agent is safe and/or efficacious for the treatment of a wound in, for example, humans.

Also provided herein are compositions for inducing the formation of a wound model in an animal. Disclosed herein is a composition comprising a scaffold and a senescence-inducing agent. Disclosed herein is a composition comprising one or more polymers and a senescence-inducing agent. Disclosed herein is a composition for inducing the formation of a wound model in an animal, the composition comprising one or more polymers and a senescence-inducing agent.

Provided herein is also a method of preparing a composition as defined herein. The method may comprise mixing (or soaking) one or more polymers with a senescence-inducing agent. The method may comprise mixing (or soaking) the one or more polymers in a solution comprising a senescence inducing agent. The solution may, for example, be a Pluronic gel (e.g. a 30% Pluronic gel). The solution may comprise BCM or about 100 μM of FK866.

Disclosed herein is a dermal substitute for inducing the formation of a wound model in an animal, the dermal substitute comprising one or more polymers and a senescence-inducing agent.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### Examples

### Methods

Excisional wounds are made on the backs of rats or pigs, with a punch biopsy (6 mm in rats and 10 mm to 12 mm in pigs). An electrospun scaffold (80% PCL and 20% rat tail collagen) is then inserted into the wound. The scaffold has been soaked in an agent that is able to induce senescence. This can be either biofilm conditioned media (BCM) from Staph aureus or a small molecular drug (FK866). The wound is then covered with an occlusive dressing of parafilm followed Opsite then a sticky crepe bandage to hold the scaffolds in place. For the first three days, the senescence inducing agent is reapplied to the wound. After five or ten days the scaffolds are removed, and the wounds are allowed to heal.

Acute wounds of these sizes on both rats and pigs normally would be fully closed in under ten days. The perturbed wounds generated from our model, however, heal at a much slower rate as well as exhibit known characteristics of human chronic wounds.

These wounds form granulation tissue quite rapidly but the epidermis remains hyper thickened for a considerable distance around the wound. Epithelial cells at the wound edge fail to migrate and it was observed that these wounds remained open even after 10 (for rats) or 30-35 (for pigs) days post scaffold removal. These cells also show a massive upregulation of Cx43 which is seen in human chronic wounds and is known to inhibit cell migration. Inflammation remains high within the granulation tissue and surrounding intact tissues, which are packed with neutrophils and macrophages. Giant cells can be seen where macrophages are fusing together. The blood vessels in the upper dermis are highly inflamed and many neutrophils can be seen to be extravasating. In these regions the extracellular matrix can be seen to begin to be degraded. Within the granulation tissue and surrounding intact tissues many of the cells are positive for a marker of senescence.

### CONCLUSIONS

The inventors have the unexpected finding that rat tail collagen would induce a severe inflammatory reaction in rat wounds. It was thought that rat tail collagen would be very biocompatible as it came from a rat. This also caused a hyper thickening of the wound edge epidermis, which failed to migrate and expressed high levels of Cx43 (as seen in human chronic wounds) which is known to inhibit migration.

The study next examined the effects of biofilm conditioned media (BCM) on wound healing. The media could be toxic but if cells survived, they could change their appearance, grow larger and flatter and accumulated granules in the cytoplasm. It turned out that the cells become senescent. Including BCM in the scaffold induced senescence in and around the wound in fibroblasts, endothelial cells and leukocytes. Including a senescence inducing drug had similar effects.

In a set of experiments, the wounds were covered with parafilm, which is non-breathable, to keep a drug in place. This had the unexpected effect of inducing hyper thickening of the epidermis and a failure of cell migration. Putting parafilm over the scaffold in the wound caused the hyper thickening to be worse.

Combining all of these treatments mimicked several of the features of human chronic wounds. The inventors did not expect wound closure to be affected as much as it has been open 30-35 days after scaffold removal.

### REFERENCE

Ewald J A et al., Therapy-induced senescence in cancer. J Natl Cancer Inst 102(20):1536-1546 (2010)
Mikula-Pietrasik, J. et al., Mechanisms and significance of therapy-induced and spontaneous senescence of cancer cells. Cell. Mol. Life Sci. 77, 213-229 (2020).

The invention claimed is:

1. A method for generating a wound model in an animal, the method comprising:
a) contacting a wound with a composition comprising a scaffold and a senescence-inducing agent.

2-19. (canceled)

20. The method of claim 1, wherein the wound model is a perturbed wound model.

21. The method of claim 1, wherein the scaffold comprises one or more polymers.

22. The method of claim 21, wherein the scaffold is electrospun.

23. The method of claim 1, wherein the one or more polymers comprise or consist of collagen.

24. The method of claim 1, wherein the one or more polymers comprise or consist of PCL and collagen.

25. The method of claim 24, wherein the one or more polymers comprise or consist 80% w/w PCL and 20% w/w collagen.

26. The method of claim 24, wherein the collagen is rat collagen.

27. The method of claim 1, wherein the senescence-inducing agent is biofilm conditioned media (BCM).

28. The method claim 1, wherein the senescence-inducing agent is a nicotinamide phosphoribosyltransferase inhibitor.

29. The method of claim 28, wherein the nicotinamide phosphoribosyltransferase inhibitor is Daporinad (FK866).

30. The method of claim 1, wherein the method comprises generating a wound in the animal prior to step (a).

31. The method of claim 30, wherein the wound is generated by a punch biopsy.

32. The method of claim 1, wherein the method comprising sealing the wound.

33. The method of claim 1, wherein the method comprises contacting the wound with the composition once a day over three days.

34. A method of identifying a candidate therapeutic agent for treating a wound, the method comprising:
a) generating a wound model in an animal according to a method of claim 1, and
b) contacting the wound model with the candidate therapeutic agent.

35. A composition for inducing the formation of a wound model in an animal, the composition comprising a scaffold and a senescence-inducing agent.
